(19) United States
(12) Patent Application Publication
(10) Pub. No.: US 2025/0228674 A1
(43) Pub. Date: Jul. 17, 2025

(54) HIP IMPINGEMENT AND DISLOCATION DETECTION SYSTEM

(71) Applicant: Orthosoft ULC, Montreal (CA)

(72) Inventor: Jerome Casaubon, Montreal (CA)

(21) Appl. No.: 18/965,518

(22) Filed: Dec. 2, 2024

Related U.S. Application Data
(60) Provisional application No. 63/621,405, filed on Jan. 16, 2024.

(51) Int. Cl. A61F 2/46; A61F 2/30

(52) U.S. Cl. CPC A61F 2/4657; A61F 2/30; A61F 2/4684; A61F 2002/30698

(57) ABSTRACT

A trial implant for detecting impingement or dislocation intraoperatively during a range of motion test can include a trial head of a ball and socket joint replacement prosthesis. The ball and socket joint replacement prosthesis can be configured to attach to a stem component and engage with a socket portion of the ball and socket joint replacement prosthesis. A sensor can be embedded within the trial head and configured to generate a signal indicative of a distance between the trial head and the socket portion. The sensor can include an inductive coil to detect the distance and generate the signal indicative of the distance.

### CLAIM OF PRIORITY

This application claims the benefit of U.S. Provisional Patent Application Ser. No. 63/621,405, filed on Jan. 16, 2024, the benefit of priority of which is claimed hereby, and which is incorporated by reference herein in its entirety.

### TECHNICAL FIELD

Examples described herein generally relate to a system. More specifically, the examples described herein relate to a hip impingement and dislocation detection system.

### BACKGROUND

The field of orthopedic surgery has long been concerned with the successful implantation of prosthetic devices to replace or support damaged joints, particularly in the hip area. Hip replacement surgery, also known as hip arthroplasty, involves replacing a diseased or damaged hip joint with an artificial implant. This procedure is commonly performed to relieve pain and improve mobility in patients suffering from conditions such as osteoarthritis, rheumatoid arthritis, osteonecrosis, or other degenerative joint diseases.

A critical aspect of hip replacement surgery is ensuring the proper fit and placement of the prosthetic components to avoid postoperative complications. One such complication is impingement, which occurs when the artificial components of the hip joint, or the prosthetic components and the patient's bone, come into abnormal contact. This can lead to pain, reduced range of motion, and wear of the prosthetic materials. Another serious complication is dislocation, where the ball component of the implant becomes dislodged from the socket, leading to immediate loss of joint function and necessitating urgent medical attention.

### SUMMARY

In examples, a system for detecting impingement or dislocation of a prosthetic implant installed within a patient, the prosthetic implant can include an implant and an articulating component. The implant can be configured to receive the head component and installed into a first bone. The articulating component can be installed into a second bone. The system can also include a trial head configured to be installed on the implant to engage with the articulating component. The trial head can include a sensor configured to generate a signal indicative of a distance between the trial head and the articulating component. The system can also include processing circuitry to receive the signal from the sensor, compare the signal from the sensor to a set threshold value, and communicate an alert on condition that the signal exceeds the set threshold value. The alert can be indicative of an impingement between the trial head and the second bone or the articulating component or a dislocation between the trial head and the articulating component.

In examples, a trial implant for detecting impingement or dislocation intraoperatively during a range of motion test can include a trial head of a ball and socket joint replacement prosthesis. The ball and socket joint replacement prosthesis can be configured to attach to a stem component and engage with a socket portion of the ball and socket joint replacement prosthesis. A sensor can be embedded within the trial head and configured to generate a signal indicative of a distance between the trial head and the socket portion. The sensor can include an inductive coil to detect the distance and generate the signal indicative of the distance.

This summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 illustrates a perspective view of an example of an impingement and dislocation detection system.

FIG. 2 illustrates an enlarged view of an example of an impingement and dislocation detection system.

FIG. 3A illustrates a perspective view of an example trial head of an impingement and dislocation detection system.

FIG. 3B illustrates an exploded view of an example trial head of an impingement and dislocation detection system.

FIG. 4 illustrates a schematic diagram of an example impingement and dislocation detection system.

FIG. 5A illustrates a diagrammatic drawing of an example prosthetic-to-prosthetic impingement of a prosthetic implant.

FIG. 5B illustrates a diagrammatic drawing of an example prosthetic-to-bone impingement of a prosthetic implant.

FIG. 5C illustrates a diagrammatic drawing of an example dislocation of a prosthetic implant.

FIG. 6 illustrates a graphical representation of an example signal during a range of motion test with an example impingement and dislocation detection system.

FIG. 7 illustrates a block diagram illustrating an example of a machine upon which one or more examples may be implemented.

FIG. 8 illustrates a schematic diagram of an example of a method.

### DETAILED DESCRIPTION

The detection of impingement and dislocation for prosthetic implants has been based on clinical examination, patient symptoms, and imaging studies post operatively. However, these methods may not provide immediate feedback during surgery, when the surgeon can still adjust the installation of the prosthetic implant, or accurately predict the risk of future complications. The development of systems to detect impingement and dislocation intraoperatively could provide significant benefits. Such systems could potentially allow for real-time adjustments during surgery, reducing the likelihood of postoperative complications and improving patient outcomes. Despite the advances in surgical techniques and prosthetic design, there remains a need for improved methods and systems to detect and prevent impingement and dislocation during hip replacement surgeries.

The present disclosure relates to an intraoperative system to detect impingement and dislocation in prosthetic implants installed into the patient. The system includes a passive sensor, which does not require power and thus can be sanitized, within a trial head that can be attached to an implant installed into a first bone of the patient. The trial head can be engaged with an articulating component implanted into a second bone of the patient. The implant, the first bone, and the second bone can together make the prosthetic joint. The medical professionals can complete an intraoperative range of motion test while the trial head is detecting changes to a distance between the trial head and the articulating component. If the change in distance between the trial head and the articulating component is above a set threshold, the system for detecting impingements and dislocations can generate an alert to notify the medical professionals of the anomaly.

The above discussion is intended to provide an overview of subject matter of the present patent application. It is not intended to provide an exclusive or exhaustive explanation of the invention. The description below is included to provide further information about the present patent application.

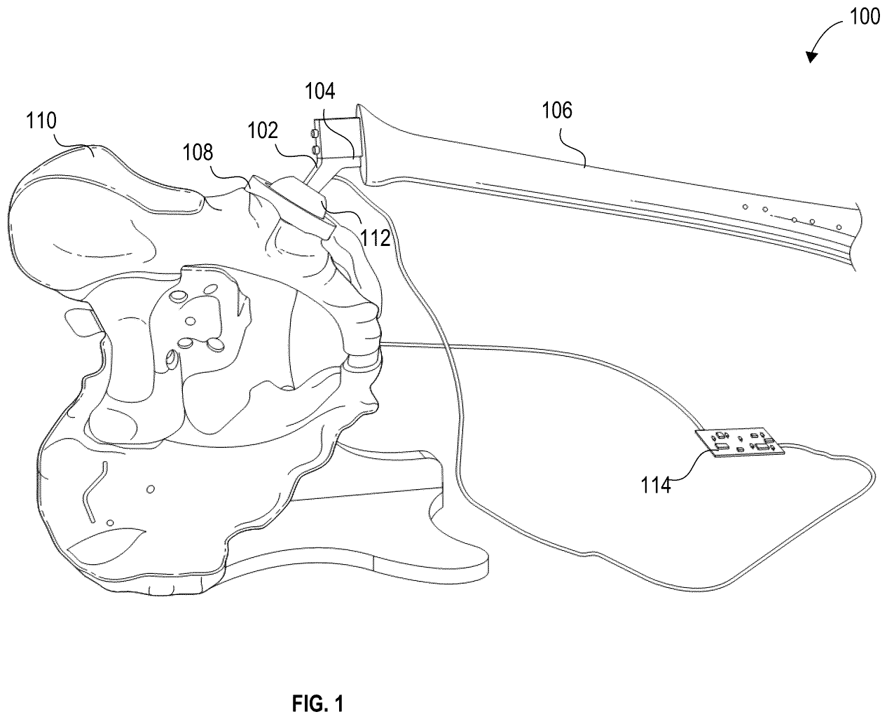

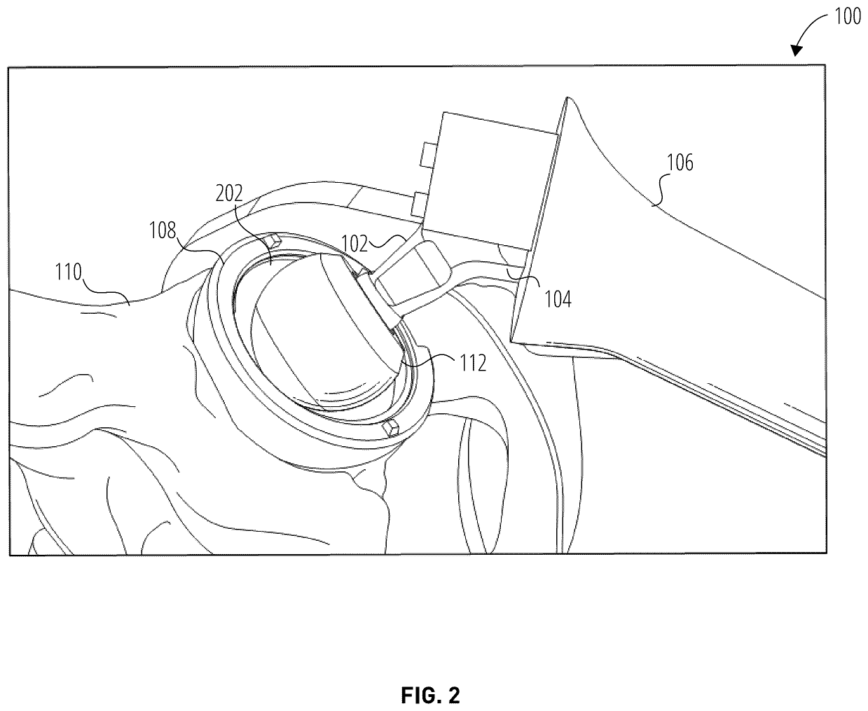

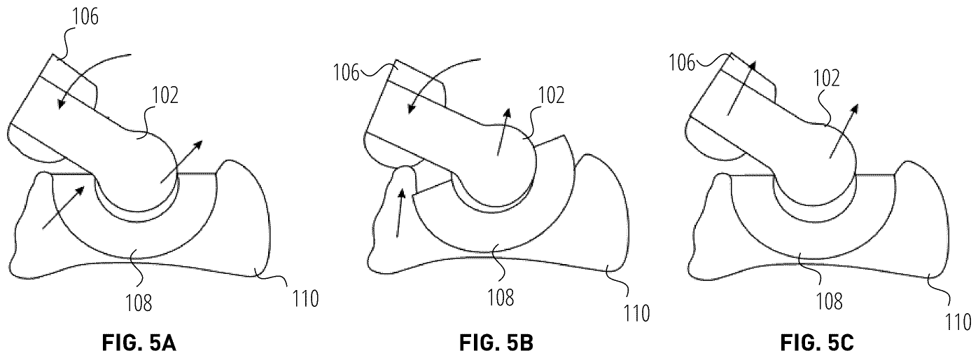

FIG. 1 and FIG. 2 will be discussed together. FIG. 1 illustrates a perspective view of an example of an impingement and dislocation detection system 100. FIG. 2 illustrates an enlarged view of an example of the impingement and dislocation detection system 100. The impingement and dislocation detection system 100 can be configured to detect an impingement (e.g., either prosthetic implant-to-bone impingement or a prosthetic-to-prosthetic impingement) or a dislocation, which will all be discussed herein with reference to FIG. 5A, FIG. 5B, and FIG. 5C. The impingement and dislocation detection system 100 can include a prosthetic implant 102, an articulating component 108, a trial head 112, and external circuitry 114.

The stem component 104 can be configured to be inserted into a first bone 106. The stem component 104 can be a permanent implant that can be configured to receive either the trial head 112 or a prosthetic head (e.g., a prosthetic femoral head for extended use within the joint to replace damaged or diseased joint surfaces). In examples, the stem component 104 can be inserted into the stem component 104 without either of the trial head 112 or the prosthetic head component installed thereon. After installation into the bone, the trial head 112 can be installed onto the stem component 104 in preparation for an intraoperative range of motion test. The examples shown in FIG. 1 include the stem component 104. In other examples, the prosthetic implant 102 can include a stemless component to receive the trial head 112.

The articulating component 108 can be configured to be inserted into the second bone 110. The articulating component 108 can be configured to engage with the prosthetic head to form a prosthetic joint between the first bone 106 and the second bone 110. The articulating component 108 can also engage with the trial head 112 during the use of the impingement and dislocation detection system 100. In examples, the articulating component 108 can be installed into the second bone 110 and can be configured to receive the trial head 112 for the intraoperative range of motion test. In examples, the articulating component 108 can include conductive materials that can be detected by a sensor within the trial head. In another example, a liner can be installed on the articulating component 108 and the liner can include conductive materials detected by the sensor.

The trial head 112 can be configured to be installed on the stem component 104 after the stem component 104 and the articulating component 108 are installed in the first bone 106 and the second bone 110, respectively. The trial head 112 can engage with the articulating component 108 during the operation (e.g., the intraoperative range of motion test) of the impingement and dislocation detection system 100. As such, the trial head 112 can be inserted into the articulating component 108 and the medical professional can complete the intraoperative range of motion test. The trial head 112 can be configured to generate a signal indicative of a distance between the trial head 112 and the articulating component 108 during the intraoperative range of motion test. After the intraoperative range of motion test, the trial head 112 can be removed from the stem component 104 and the prosthetic head can be installed on the stem component 104 to engage with the articulating component 108 and form the prosthetic joint with the articulating component 108. In examples, the trial head 112 can be a single-use product that is used for a single range of motion test. In another example, the trial head 112 can be recycled, refurbished, or reconditioned for multiple uses.

The external circuitry 114 can be configured to receive the signal from the trial head 112. The external circuitry 114 can process the signal and determine whether an impingement or dislocation occurs during the intraoperative range of motion test. The external circuitry 114 can analyze the signals and send a summary to the medical professional during the intraoperative range of motion test or after the medical procedure is completed. The summary can be helpful intra-operatively to provide guidance to the medical professionals with information to help inform required adjustments to avoid impingements or dislocations of the prosthetic implant. The summary can also help the medical professional provide a rehab treatment plan and post-procedure guidelines to the patient.

As shown in FIG. 2, the articulating component 108 can also include a liner 202. In some examples, the articulating component 108 can be made from conductive materials, which can be detected via the trial head 112. In other examples, the liner 202 can include conductive materials, which can be detected via the trial head 112. The liner 202 can be integrated into the articulating component 108 such that it remains attached to the articulating component 108 during the life of the impingement and dislocation detection system 100. In other examples, the liner 202 can be installed in preparation for the range of motion test and be removed after the range of motion test is completed. If the liner 202 is only for the range of motion test, the liner 202 must be thin such that it has minimal effects on the interactions between the prosthetic implant 102 (e.g., the trial head 112 or the prosthetic head) and the articulating component 108 during the range of motion test to ensure the range of motion tests are accurate and can mimic post-operative operation of the prosthetic implant 102.

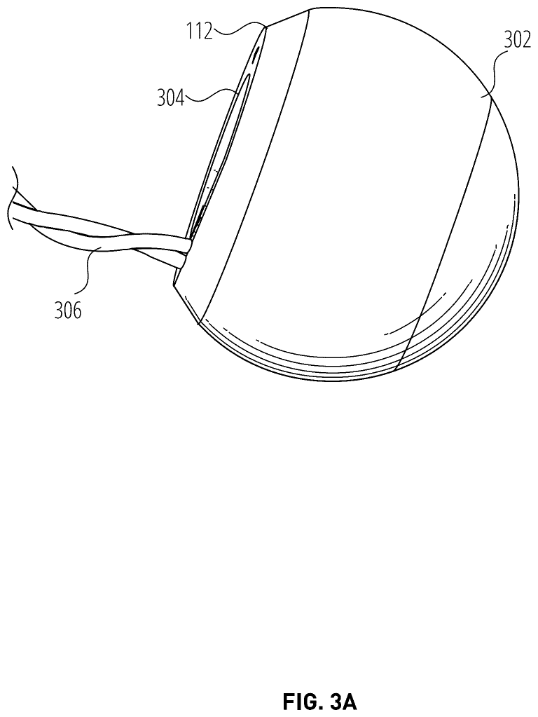

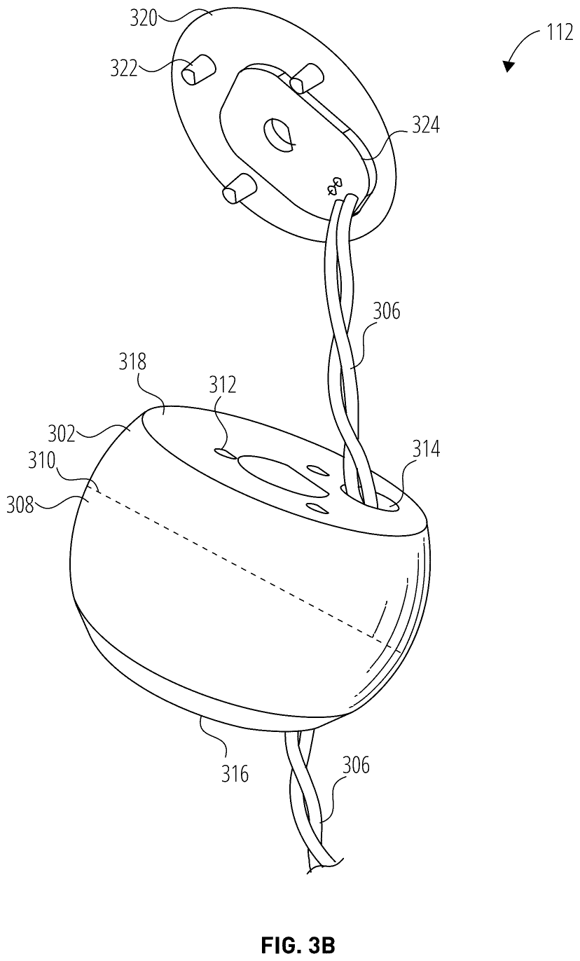

FIG. 3A illustrates a perspective view of an example trial head 112 of an impingement and dislocation detection system 100. The trial head 112 can include a housing 302, a coupling interface 304, and a wire 306 (or pair of wires). In examples, the wire can be a flexible printed circuit board (PCB).

The housing 302 can be configured to protect the sensor (e.g., sensor 324 (FIG. 3B)) and simulate the prosthetic head of the prosthetic implant such that the trial head 112 can determine how the prosthetic head will engage with the articulating component 108 after installation thereon the stem component 104. As such, the housing 302 can be sized to match a size of the planned prosthetic head that will be installed onto the stem component 104. The housing can be made from a polymer, non-ferrous metal, composite, a combination or alloy thereof, or the like.

The coupling interface 304 can be formed into the housing 302. The coupling interface 304 can be configured to receive the stem component 104 to removably couple the trial head 112 to the stem component 104. In examples, such as shown in FIG. 3A, the coupling interface 304 can be a female adapter for coupling the trial head 112 to the stem component 104. In other examples, the coupling interface 304 can be a male adapter configured to be inserted into the stem component 104 to couple the trial head 112 to the stem component 104.

The wire 306 can be configured to communicate the signal from the sensor within the trial head 112 to the external circuitry 114. The wire 306 can extend between the external circuitry 114 and the trial head 112. As shown in FIG. 3A, the wire 306 can extend into the housing 302.

FIG. 3B illustrates an exploded view of an example trial head 112. The trial head 112 can also include a first portion 308, a second portion 320, an engagement interface 322, and a sensor 324.

The first portion 308 can extend between the proximal surface 316 and the distal surface 318. The first portion 308 can define the diameter 310 of the trial head 112. As discussed herein, the trial head 112 can be configured to be the same size as the prosthetic head that is planned to be installed onto the stem component 104 (FIG. 1). Therefore, the impingement and dislocation detection system 100 (FIG. 1) can include different sizes of the trial head 112, each trial head 112 of the trial heads 112 can include a different diameter 310. Thus, preoperatively, the trial head 112 with the same diameter as the prosthetic head can be selected.

The first portion 308 can include a coupling interface 312. As shown in FIG. 3B, the coupling interface 312 can be a female connection adapter configured to receive a portion of the second portion 320 to help couple the first portion 308 and the second portion 320. In another example, the coupling interface 312 can be a male adapter configured to engage with the second portion 320 to couple the first portion 308 and the second portion 320.

The first portion 308 can include a wire channel 314 configured to receive the wire 306 therethrough. The wire channel 314 can extend through the diameter 310 such that the first portion 308 can be connected to the sensor 324 and extend outside of the trial head 112. To limit interference with the range of motion test, the wire channel 314 can extend from the distal surface 318 to the proximal surface 316 such that the wire 306 extends therefrom the proximal surface 316 and to the external circuitry 114.

The second portion 320 can be configured to be removably coupled to the first portion 308. The second portion 320 can be configured to mount the sensor 324 within the housing 302 when the second portion 320 is attached to the first portion 308.

The second portion 320 can include an engagement interface 322. The engagement interface 322 can be complementary with the coupling interface 312 of the first portion 308 such as to aid in the coupling of the first portion 308 and the second portion 320. Moreover, the coupling interface 312 and the engagement interface 322 can include a pattern such as to ensure the first portion 308 and the second portion 320 can only be attached in a single position. As such, the sensor 324 can be located in a predictable location and orientation within the second portion 320 and the wire 306 extending from the sensor 324 can be aligned with the wire channel 314 of the first portion 308.

The sensor 324 can be attached to the second portion 320 and encompassed by the first portion 308 and the second portion 320 when the first portion 308 is coupled to the second portion 320. The sensor 324 can be an inductive coil configured to detect a distance away from the articulating component 108 during the intraoperative range of motion test. For example, the sensor 324 can detect a distance between the sensor 324 and the conductive material of the articulating component 108. The sensor 324 can also detect a distance between the sensor 324 and a liner, which can include inductive materials. As discussed herein, the liner can be installed therein the articulating component 108. The sensor 324 can also include an accelerometer, other proximity sensor, gyro-sensor, any combination thereof, or the like. As shown in FIG. 3B, the sensor 324 can be mounted to the second portion 320. In another example, the distal surface 318 can be configured to receive the sensor 324 such that the sensor 324 is installed within the first portion 308 and the second portion 320 is configured to hold the sensor 324 within the first portion 308. the sensor 324 can be a flexible printed circuit board (PCB). In another example, the sensor 324 can be integral to the PCB of the wire 306.

In the examples shown in FIG. 3B, the housing 302 is a two-piece housing, which can be configured to receive the sensor 324 (e.g., either within the second portion 320 (as shown in FIG. 3B) or within the first portion 308 (as discussed above). In other examples, the housing 302 can be a single monolithic component and the sensor 324 can be integral to the housing 302. For example, the housing 302 can be additively manufactured around the sensor 324 such that the housing 302 includes the sensor 324 installed within the single-piece version of the housing 302. In another example, the housing 302 can include more than two pieces coupled together. Such a design can be more adjustable as to where the sensor 324 mounts within the housing 302 and how the trial head 112 matches the prosthetic head of the prosthetic implant 102 (FIG. 1).

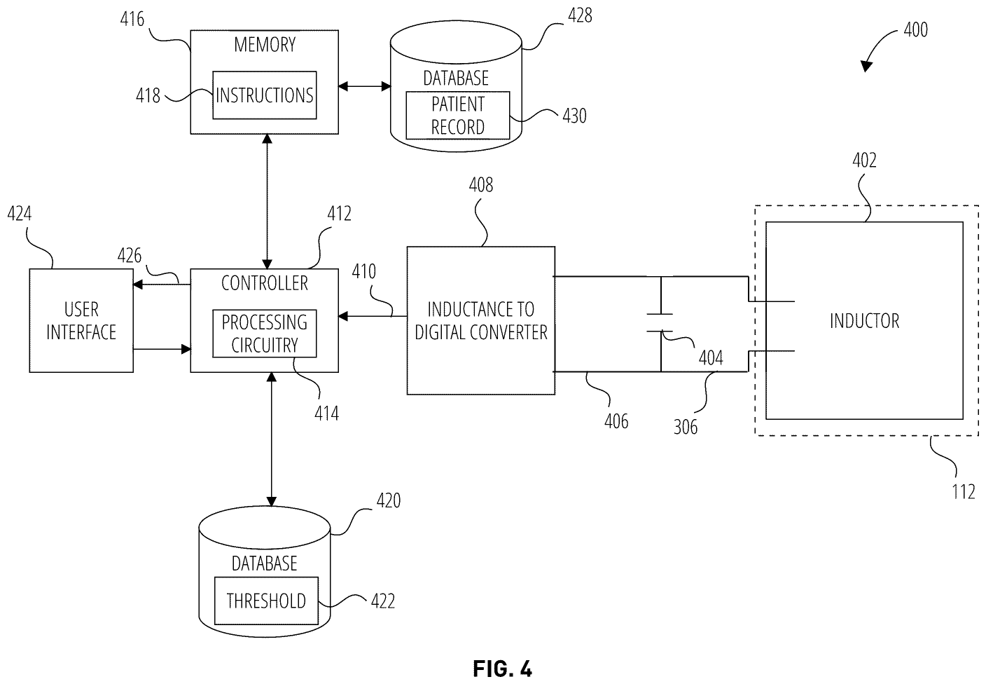

FIG. 4 illustrates a schematic diagram of an example impingement and dislocation detection system 400 (e.g., the impingement and dislocation detection system 100). The impingement and dislocation detection system 400 can include an inductor 402, a capacitor 404, an inductance to digital converter 408, a controller 412, a memory 416, a database 420, a user interface 424, an alert 426, and a database 428.

The capacitor 404 can be connected to the inductive coil (e.g., the inductor 402). The capacitor 404 and the inductor 402 can be configured to generate a resonant frequency 406. The resonant frequency 406 can be inversely related to a distance between the trial head 112 and the socket portion (e.g., the articulating component 108 or the liner 202) such that the resonant frequency 406 decreases as the distance between the trial head 112 and the socket portion (e.g., the articulating component 108 or the liner 202) increases.

The inductance to digital converter 408 can be configured to generate the signal 410 in response to changes in the electrical signal received from the capacitor 404. In other words, the inductance to digital converter 408 can be configured to convert the resonate resonant frequency 406 to a digital value (e.g., the signal 410) for use by the controller 412 (or other components of the impingement and dislocation detection system 400). The inductance to digital converter 408 can communicate the signal 410 to the controller 412.

The controller 412 can include processing circuitry 414. The controller 412 can be coupled to a memory 416 including instructions 418. The controller 412 can also be coupled to the database 420 including a threshold 422. The controller 412 can be configured to receive the signal 410 and analyze the signal 410. The instructions 418, when initiated by the processing circuitry 414, can configure the processing circuitry 414 to receive the signal 410, compare the signal 410 from the sensor 324 to set threshold values (e.g., threshold 422), and communicate an alert (e.g., alert 426) on condition that the signal 410 exceeds the threshold 422. The alert 426 can be indicative of an impingement between the trial head 112 (FIG. 1) and the articulating component 108 or the trial head 112 and the bone (e.g., the second bone 110 (FIG. 1)) of the patient. The alert 426 can also be indicative of a dislocation of the trial head 112 relative to the articulating component 108. The alert 426 can include one or more of an audible alert, a visual indicia, or a haptic response.

The instructions 418 can also cause the processing circuitry 414 to perform operations to generate a record of the signal (e.g., the resonate resonant frequency 406 or the signal 410) obtained from the sensor (e.g., the inductor 402, or the inductance to digital converter 408) and store the record in a database (e.g., the database 428). More specifically, the signals (e.g., the resonate resonant frequency 406 or the signal 410) can be stored in the patient record 430 to be linked to the patient receiving the prosthetic implant. This sensor data can help the medical team provide rehabilitative suggestions and track the range of motion improvements as the patient recovers from the medical procedure.

FIG. 5A, FIG. 5B, and FIG. 5C are examples of impingements and dislocations that the impingement and dislocation systems (e.g., the impingement and dislocation detection system 100 and the impingement and dislocation detection system 400) are configured to detect.

FIG. 5A illustrates a diagrammatic drawing of an example prosthetic-to-prosthetic impingement of a prosthetic implant 102 and an articulating component 108. As the range of motion test is being performed, the prosthetic implant 102 rotates and contacts the articulating component 108. The contact between the prosthetic implant 102 and the articulating component 108 causes the head of the prosthetic implant 102 to lift from, and no longer contact, the articulating component 108. As discussed herein, the trial head 112 (FIG. 1) can be configured to detect that change in distance between the head and the articulating component 108 to generate a signal and the impingement and dislocation detection system 100 (FIG. 1) (or the impingement and dislocation detection system 400 (FIG. 4)) can use that signal and generate an alert 426 if the change is over a set threshold value (e.g., threshold 422 (FIG. 4)).

FIG. 5B illustrates a diagrammatic drawing of an example prosthetic-to-bone impingement of a prosthetic implant 102 and a second bone 110 of the patient. As the range of motion test is being performed, the prosthetic implant 102 rotates and contacts the second bone 110. The contact between the prosthetic implant 102 and the second bone 110 causes the head of the prosthetic implant 102 to lift from, and no longer contact, the articulating component 108. As discussed herein, the trial head 112 (FIG. 1) can be configured to detect that change in distance between the head and the articulating component 108 to generate a signal and the impingement and dislocation detection system 100 (FIG. 1) (or the impingement and dislocation detection system 400 (FIG. 4)) can use that signal and generate an alert 426 if the change is over a set threshold value (e.g., threshold 422 (FIG. 4)).

FIG. 5C illustrates a diagrammatic drawing of an example dislocation of a prosthetic implant 102 and an articulating component 108. During the range of motion test, rotation of the prosthetic implant 102 relative to the articulating component 108 can cause the prosthetic implant 102 and the articulating component 108 to lose contact and no longer engage with one another causing a gap between the articulating component 108 and the prosthetic implant 102. As discussed herein, the trial head 112 (FIG. 1) can be configured to detect that change in distance between the head and the articulating component 108 to generate a signal and the impingement and dislocation detection system 100 (FIG. 1) (or the impingement and dislocation detection system 400 (FIG. 4)) can use that signal and generate an alert 426 if the change is over a set threshold value (e.g., threshold 422 (FIG. 4)).

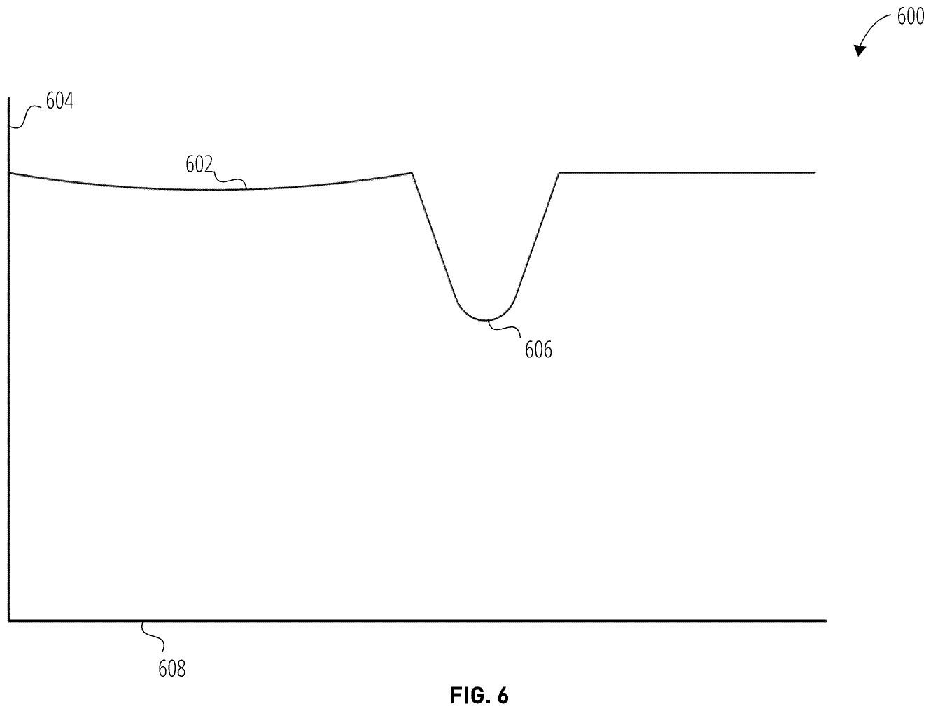

FIG. 6 illustrates a graphical representation of an example signal 602 (e.g., resonate resonant frequency 406 or signal 410) during a range of motion test with an example impingement and dislocation detection system (e.g., the impingement and dislocation detection system 100 (FIG. 1) or the impingement and dislocation detection system 400 (FIG. 4)). As shown in the graphical representation 600, the signal 602 can be plotted using a magnitude 604 of the signal through time 608.

The impingement and dislocation detection system (e.g., the impingement and dislocation detection system 100 (FIG. 1)) or the impingement and dislocation detection system 400 (FIG. 4)) can detect an anomaly in the signal 602 as the signal 602 approaches the minimum 606 because there was a change in the signal that caused the minimum 606. In response to the minimum 606 detected by the impingement and dislocation detection system, the impingement and dislocation detection system can generate an alert to notify medical professionals of the anomaly.

In an example, the impingement and dislocation detection system (e.g., the impingement and dislocation detection system 100 (FIG. 1) or the impingement and dislocation detection system 400 (FIG. 4)) can be designed such that the magnitude 604 of the signal 602 reduces as the induction sensor (e.g., the sensor 324 (FIG. 3)) is moved away from the liner or insert of the cup (e.g., the induction signal reduces when displacement of the trial head (e.g., the trial head 112) out of the cup (e.g., the articulating component 108) occurs). In other words, the signal 602 can include an inverse relationship to the distance between the trial head 112 and the articulating component 109.

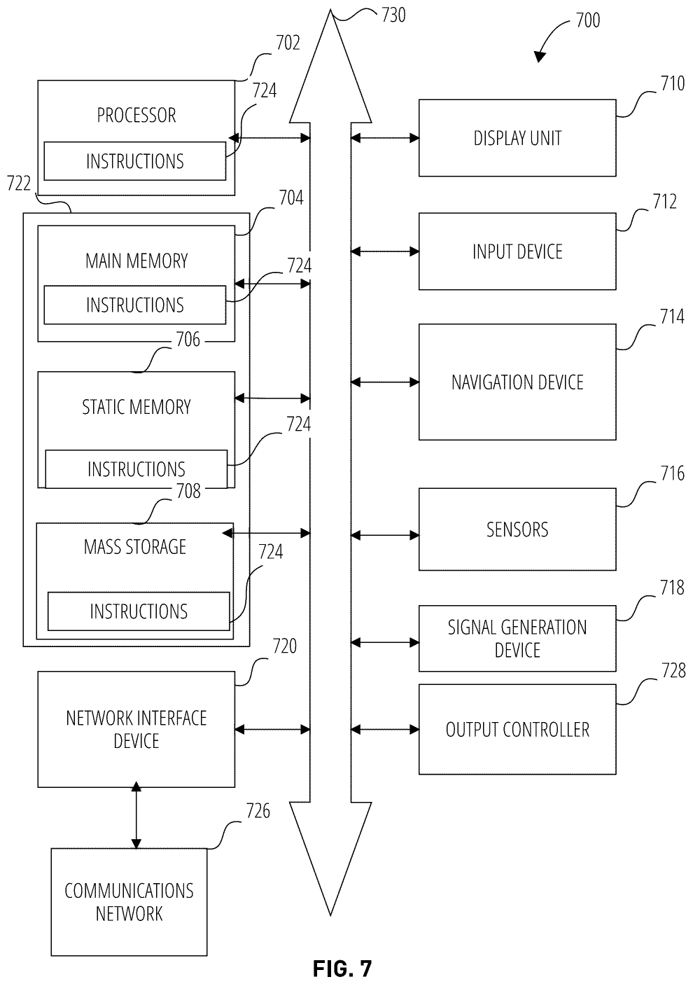

FIG. 7 illustrates a block diagram of an example machine 700 upon which any one or more of the techniques (e.g., methodologies) discussed herein may perform. Examples, as described herein, may include, or may operate by, logic or a number of components, or mechanisms in the machine 700. Circuitry (e.g., processing circuitry) is a collection of circuits implemented in tangible entities of the machine 700 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 700 follow.

In alternative examples, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 700 may include a hardware processor 702 (e.g., a central processing unit (CPU), a graphics processing unit (GPU), a hardware processor core, or any combination thereof), a main memory 704, a static memory (e.g., memory or storage for firmware, microcode, a basic-input-output (BIOS), and mass storage 708 (e.g., hard drives, tape drives, flash storage, or other block devices) some or all of which may communicate with each other via an interlink 530 (e.g., bus). The machine 700 may further include a display unit 710, an alphanumeric input device 712 (e.g., a keyboard), and a user interface (UI) navigation device 714 (e.g., a mouse). In an example, the display unit 710, input device 712 and UI navigation device 714 may be a touch screen display. The machine 700 may additionally include a signal generation device 718 (e.g., a speaker), a network interface device 720, and one or more sensors 716, such as a global positioning system (GPS) sensor, compass, accelerometer, or other sensor. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 702, the main memory 704, the static memory 706, or the mass storage 708 may be, or include, a machine readable medium 722 on which is stored one or more sets of data structures or instructions 724 (e.g., software) embodying or utilized by any one or more of the techniques or functions described herein. The instructions 724 may also reside, completely or at least partially, within any of registers of the processor 702, the main memory 704, the static memory 706, or the mass storage 708 during execution thereof by the machine 700. In an example, one or any combination of the hardware processor 702, the main memory 704, the static memory 706, or the mass storage 708 may constitute the machine readable media 722. While the machine readable medium 722 is illustrated as a single medium, the term “machine readable medium” may include a single medium or multiple media (e.g., a centralized or distributed database, and/or associated caches and servers) configured to store the one or more instructions 724.

The term “machine readable medium” may include any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine readable medium 722 may be representative of the instructions 724, such as instructions 724 themselves or a format from which the instructions 724 may be derived. This format from which the instructions 724 may be derived may include source code, encoded instructions (e.g., in compressed or encrypted form), packaged instructions (e.g., split into multiple packages), or the like. The information representative of the instructions 724 in the machine readable medium 722 may be processed by processing circuitry into the instructions to implement any of the operations discussed herein. For example, deriving the instructions 724 from the information (e.g., processing by the processing circuitry) may include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 724.

In an example, the derivation of the instructions 724 may include assembly, compilation, or interpretation of the information (e.g., by the processing circuitry) to create the instructions 724 from some intermediate or preprocessed format provided by the machine readable medium 722. The information, when provided in multiple parts, may be combined, unpacked, and modified to create the instructions 724. For example, the information may be in multiple compressed source code packages (or object code, or binary executable code, etc.) on one or several remote servers. The source code packages may be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 724 may be further transmitted or received over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 702.11 family of standards known as Wi-Fi®, IEEE 702.15.4 family of standards, peer-to-peer (P2P) networks, among others. In an example, the network interface device 720 may include one or more physical jacks (e.g., Ethernet, coaxial, or phone jacks) or one or more antennas to connect to the communications network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 700, and includes digital or analog communications signals or other intangible medium to facilitate communication of such software. A transmission medium is a machine-readable medium.

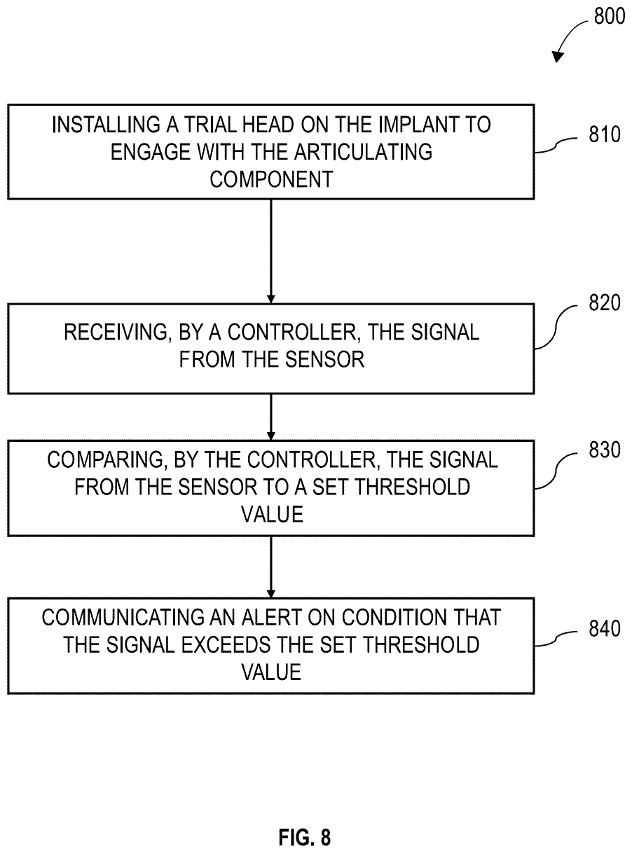

FIG. 8 illustrates a schematic diagram of an example of a method 800. The method 800 can be for detecting impingement or dislocation of a prosthetic implant installed within a patient. The prosthetic implant can include an implant configured to receive a head component and installed into a first bone and an articulating component installed into a second bone. The method 800 can optionally include operations 810-840.

At operation 810, the method 800 can include installing a trial head on the implant to engage with the articulating component. The trial head can include a sensor configured to generate a signal indicative of a distance between the trial head and the articulating component.

At operation 820, the method 800 can include receiving, by a controller, the signal from the sensor.

At operation 830, the method 800 can include comparing, by the controller, the signal from the sensor to a set threshold value.

At operation 840, the method 800 can include communicating an alert on condition that the signal exceeds the set threshold value. The alert can be indicative of an impingement between the trial head and the second bone or the articulating component or a dislocation between the trial head and the articulating component.

The following, non-limiting examples, detail certain aspects of the present subject matter to solve the challenges and provide the benefits discussed herein, among others.

Example 1 is a system for detecting impingement or dislocation of a prosthetic implant installed within a patient, the prosthetic implant including an implant configured to receive a head component and installed into a first bone and an articulating component installed into a second bone, the system comprising: a trial head configured to be installed on the implant to engage with the articulating component, the trial head including: a sensor configured to generate a signal, the signal indicative of a distance between the trial head and the articulating component; a memory including instructions; and a controller coupled to the memory including instructions, the instructions configured to, when executed by processing circuitry of the controller, cause the processing circuitry to perform operations including: receive the signal from the sensor; compare the signal from the sensor to a set threshold value; and communicate an alert on condition that the signal exceeds the set threshold value, the alert indicative of an impingement between the trial head and the second bone or the articulating component or a dislocation between the trial head and the articulating component.

In Example 2, the subject matter of Example 1 optionally includes wherein the sensor comprises: an inductive coil; and a capacitor connected to the inductive coil, the capacitor and the inductive coil configured to generate a resonant frequency, the resonant frequency is inversely related to the distance between the trial head and the articulating component such that the resonant frequency decreases as the distance between the trial head and the articulating component increases.

In Example 3, the subject matter of Example 2 optionally includes wherein the sensor comprises: an inductance to digital converter, the inductance to digital converter configured to convert the resonant frequency to a digital value for use by the system.

In Example 4, the subject matter of any one or more of Examples 1-3 optionally include wherein the sensor is passive and requires no power to operate.

In Example 5, the subject matter of any one or more of Examples 1-4 optionally include wherein the articulating component includes a liner, the liner including a conductive material.

In Example 6, the subject matter of Example 5 optionally includes wherein the sensor is configured to detect a distance between the trial head and the conductive material of the liner.

In Example 7, the subject matter of any one or more of Examples 1-6 optionally include wherein the alert can include one or more of an audible alert, a visual indicia, or a haptic response.

In Example 8, the subject matter of any one or more of Examples 1-7 optionally include wherein the instructions cause the processing circuitry to perform operations including: generate a record of the signal obtained from the sensor; and save the record of the signal obtained from the sensor in a database.

In Example 9, the subject matter of Example 8 optionally includes wherein the database includes a patient record database, the patient record database including patient-specific medical information including at least the signal obtained from the sensor during a range of motion test.

Example 10 is a trial implant for detecting impingement or dislocation of a prosthetic implant installed within a patient intraoperatively during a range of motion test, the trial implant including: a trial head of a ball and socket joint replacement prosthesis, the ball and socket joint replacement prosthesis configured to attach to a stem component and engage with a socket portion of the ball and socket joint replacement prosthesis; and a sensor embedded within the trial head, the sensor configured to generate a signal indicative of a distance between the trial head and the socket portion, the sensor including an inductive coil to detect the distance and generate the signal indicative of the distance.

In Example 11, the subject matter of Example 10 optionally includes wherein the sensor comprises: a capacitor connected to the inductive coil, the capacitor and the inductive coil configured to generate a resonant frequency, the resonant frequency is inversely related to the distance between the trial head and the socket portion such that the resonant frequency decreases as the distance between the trial head and the socket portion increases.

In Example 12, the subject matter of Example 11 optionally includes wherein the sensor comprises: an inductance to digital converter, the inductance to digital converter configured to convert the resonant frequency to a digital value for use by the trial implant.

In Example 13, the subject matter of any one or more of Examples 10-12 optionally include wherein the sensor is passive and requires no power to operate.

In Example 14, the subject matter of any one or more of Examples 10-13 optionally include wherein the socket portion includes a liner, the liner including a conductive material.

In Example 15, the subject matter of Example 14 optionally includes wherein the sensor is configured to detect a distance between the trial head and the conductive material of the liner.

In Example 16, the subject matter of any one or more of Examples 10-15 optionally include a controller including processing circuitry and coupled to a memory, the memory including instructions that, when executed by the processing circuitry, cause the processing circuitry to: obtain the signal from the sensor; compare the signal from the sensor to a set threshold value; and communicate an alert on condition that the signal exceeds the set threshold value, the alert indicative of an impingement between the trial head and the socket portion or a bone of the patient, or a dislocation between the trial head and the socket portion.

In Example 17, the subject matter of Example 16 optionally includes wherein the alert can include one or more of an audible alert, a visual indicia, or a haptic response.

In Example 18, the subject matter of any one or more of Examples 16-17 optionally include wherein the instructions cause the processing circuitry to: generate a record of the signal obtained from the sensor; and save the record of the signal obtained from the sensor in one or more databases.

In Example 19, the subject matter of Example 18 optionally includes wherein the one or more databases includes a patient record database, the patient record database including patient-specific medical information including at least the signal obtained from the sensor during the range of motion test.

Example 20 is a method, apparatus, or system including any element of any of Examples 1-19.

The above-detailed description includes references to the accompanying drawings, which form a part of the detailed description. The drawings show, by way of illustration, specific examples that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

What is claimed is:

1. A system for detecting impingement or dislocation of a prosthetic implant installed within a patient, the prosthetic implant including an implant configured to receive a head component and installed into a first bone and an articulating component installed into a second bone, the system comprising:
a trial head configured to be installed on the implant to engage with the articulating component, the trial head including:
a sensor configured to generate a signal, the signal indicative of a distance between the trial head and the articulating component;
a memory including instructions; and
a controller coupled to the memory including instructions, the instructions configured to, when executed by processing circuitry of the controller, cause the processing circuitry to perform operations including:
receive the signal from the sensor;
compare the signal from the sensor to a set threshold value; and
communicate an alert on condition that the signal exceeds the set threshold value, the alert indicative of an impingement between the trial head and the second bone or the articulating component or a dislocation between the trial head and the articulating component.

2. The system of claim 1, wherein the sensor comprises:
an inductive coil; and
a capacitor connected to the inductive coil, the capacitor and the inductive coil configured to generate a resonant frequency, the resonant frequency is inversely related to the distance between the trial head and the articulating component such that the resonant frequency decreases as the distance between the trial head and the articulating component increases.

3. The system of claim 2, wherein the sensor comprises:
an inductance to digital converter, the inductance to digital converter configured to convert the resonant frequency to a digital value for use by the system.

4. The system of claim 1, wherein the sensor is passive and requires no power to operate.

5. The system of claim 1, wherein the articulating component includes a liner, the liner including a conductive material.

6. The system of claim 5, wherein the sensor is configured to detect a distance between the trial head and the conductive material of the liner.

7. The system of claim 1, wherein the alert can include one or more of an audible alert, a visual indicia, or a haptic response.

8. The system of claim 1, wherein the instructions cause the processing circuitry to perform operations including:
generate a record of the signal obtained from the sensor; and
save the record of the signal obtained from the sensor in a database.

9. The system of claim 8, wherein the database includes a patient record database, the patient record database including patient-specific medical information including at least the signal obtained from the sensor during a range of motion test.

10. A trial implant for detecting impingement or dislocation of a prosthetic implant installed within a patient intraoperatively during a range of motion test, the trial implant including:
a trial head of a ball and socket joint replacement prosthesis, the ball and socket joint replacement prosthesis configured to attach to a stem component and engage with a socket portion of the ball and socket joint replacement prosthesis; and
a sensor embedded within the trial head, the sensor configured to generate a signal indicative of a distance between the trial head and the socket portion, the sensor including an inductive coil to detect the distance and generate the signal indicative of the distance.

11. The trial implant of claim 10, wherein the sensor comprises:
a capacitor connected to the inductive coil, the capacitor and the inductive coil configured to generate a resonant frequency, the resonant frequency is inversely related to the distance between the trial head and the socket portion such that the resonant frequency decreases as the distance between the trial head and the socket portion increases.

12. The trial implant of claim 11, wherein the sensor comprises:
an inductance to digital converter, the inductance to digital converter configured to convert the resonant frequency to a digital value for use by the trial implant.

13. The trial implant of claim 10, wherein the sensor is passive and requires no power to operate.

14. The trial implant of claim 10, wherein the socket portion includes a liner, the liner including a conductive material.

15. The trial implant of claim 14, wherein the sensor is configured to detect a distance between the trial head and the conductive material of the liner.

16. The trial implant of claim 10, comprising:
a controller including processing circuitry and coupled to a memory, the memory including instructions that, when executed by the processing circuitry, cause the processing circuitry to:
obtain the signal from the sensor;
compare the signal from the sensor to a set threshold value; and
communicate an alert on condition that the signal exceeds the set threshold value, the alert indicative of an impingement between the trial head and the socket portion or a bone of the patient, or a dislocation between the trial head and the socket portion.

17. The trial implant of claim 16, wherein the alert can include one or more of an audible alert, a visual indicia, or a haptic response.

18. The trial implant of claim 16, wherein the instructions cause the processing circuitry to:
generate a record of the signal obtained from the sensor; and
save the record of the signal obtained from the sensor in one or more databases.

19. The trial implant of claim 18, wherein the one or more databases includes a patient record database, the patient record database including patient-specific medical information including at least the signal obtained from the sensor during the range of motion test.

20. A method for detecting impingement or dislocation of a prosthetic implant installed within a patient, the prosthetic implant including an implant configured to receive a head component and installed into a first bone and an articulating component installed into a second bone, the method comprising:
installing a trial head on the implant to engage with the articulating component, the trial head including a sensor configured to generate a signal indicative of a distance between the trial head and the articulating component;
receiving, by a controller, the signal from the sensor;
comparing, by the controller, the signal from the sensor to a set threshold value; and
communicating an alert on condition that the signal exceeds the set threshold value, the alert indicative of an impingement between the trial head and the second bone or the articulating component or a dislocation between the trial head and the articulating component.
